(19) United States
(12) Patent Application Publication
(10) Pub. No.: US 2005/0079387 A1
(43) Pub. Date: Apr. 14, 2005

(54) Imidazole ring-containing compound and organic electroluminescence display device

(76) Inventors: Seok-Jong Lee, Suwon-si (KR); Young-Kook Kim, Suwon-si (KR); Hee-Yeon Kim, Suwon-si (KR); Seok-Hwan Hwang, Suwon-si (KR); Seung-Gak Yang, Suwon-si (KR); Young-Rag Do, Seoul (KR); Joo-Han Song, Suwon-si (KR)

Correspondence Address: MCGUIREWOODS, LLP, 1750 TYSONS BLVD, SUITE 1800, MCLEAN, VA, 22102, US

(21) Appl. No.: 10/961,202

(22) Filed: Oct. 12, 2004

(30) Foreign Application Priority Data
Oct. 13, 2003 (KR) 2003-0070988

(51) Int. Cl. H05B 33/12; C07D 498/02; C07D 513/02; C07D 517/02; C07D 235/02

(52) U.S. Cl. 428/690; 428/917; 313/504; 546/121; 548/121; 548/154; 548/218

(57) ABSTRACT

The present invention is related to an imidazole ring-containing compound and an organic electroluminescence (EL) display device using the same. In particular, the imidazole ring-containing compound has strong blue luminescence and hole transporting characteristics and may be used as a blue luminescent material and as a host of phosphorescent and fluorescent dopants in various colors such as red, green, blue, and white. In addition, an organic EL display device may be manufactured using the imidazole ring-containing compound of the present invention, which has high-efficiency luminescence characteristics and consumes less power.

### CROSS-REFERENCE TO RELATED APPLICATIONS

This application claims priority to Korean Patent Application No. 2003-70988, filed on Oct. 13, 2003, in the Korean Intellectual Property Office, the disclosure of which is incorporated herein in its entirety by reference.

### FIELD OF THE INVENTION

The present invention is related to an imidazole ring-containing compound and an organic electroluminescence (EL) display device using the same. More particularly, the present invention is related to a blue luminescent host compound and an organic EL display device using the same.

### BACKGROUND

In general, an organic EL display device is composed of an anode on the top surface of a substrate. Additionally, a hole transporting layer, an electroluminescent layer, an electron transporting layer, and a cathode may be sequentially formed on the anode. The hole transporting layer, the electroluminescent layer, and the electron transporting layer may be thin films made of organic compounds.

Organic EL display devices with the above-described structure may operate according to the following principles. When a voltage is applied across the anode and the cathode, holes injected from the anode migrate via the hole transporting layer into the electroluminescent layer. Electrons injected from the cathode migrate via the electron transporting layer into the electroluminescent layer and combine with the holes therein to generate excitons. When the excitons transit from an excited state to a base state, fluorescent molecules in the electroluminescent layer emit light to form visible images. Light emission occurring as excitons transiting from a singlet state (Si) to a base (SO) state is referred to as “fluorescence”, and light emission occurring as excitons transiting from a triplet (TI) state to a base state is referred to as “phosphorescence”. In fluorescence only 25% of the singlet state excitons (75% of triplet state excitons) are used, thereby limiting emission efficiency. In contrast, in phosphorescence, 75% of triplet state excitons and 25% of singlet state excitons are used so that 100% internal quantum efficiency,may be theoretically achieved.

A high-efficiency, green and red organic EL device has been developed using Ir(ppy)₃, which is a phosphorescent colorant having a heavy atom such as Ir or Pt with strong spin-orbit bond and PtOEP as dopants to enable effective light emission in a triplet (phosphorescent) state. In the organic EL device, CBP (4,4′-N,N′-dicarbazole-biphenyl) may be used as a host. This organic EL device, however, has a short lifespan of 150 hours because the CBP has a low glass transition temperature of 110° C. and is susceptible to crystallization, so it is unsuitable for commercial use.

### SUMMARY OF THE INVENTION

The present invention is directed to a host material suitable for fluorescent and phosphorescent dopants of any color such as red, green, blue, and white. The host material has improved electrical stability, better charge transporting capability, a high glass transition temperature, and does not crystallize. Moreover, the present invention is also directed to a high-efficiency, low-voltage, high-luminance, long-lifespan organic EL device using the host material.

According to an aspect of the present invention, there is provided an imidazole ring-containing compound of Formula (1) below:
Formula 1
   Ar₁-Ar₂-Ar₃

where Ar₂ may include, but is not limited to the compounds represented by Formula (2) below: 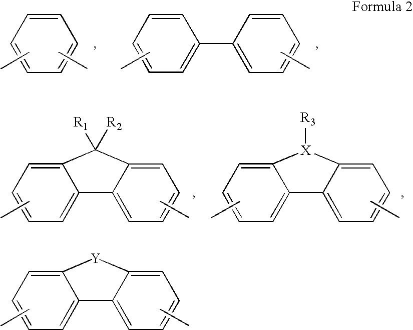
where X may be N, B, or P; Y may be O, S, or Se; and each of R₁, R₂, and R₃ may independently be a hydrogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C2-C30 heteroacryl group, a substituted or unsubstituted C5-C30 condensed polycyclic group, a heteroaryloxy group, and a substituted or unsubstituted C6-C30 condensed polycyclic group. Furthermore, R₁ and R₂ may be capable of combining together to form a saturated or unsaturated ring. Also, each of Ar₁ and Ar₃ may independently be selected from the groups represented by Formula (3), as depicted below: 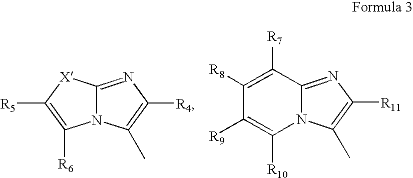
where X′ may be O, S, or Se; each of R₄ and R₁₁ may independently be selected from a hydrogen atom, a halogen atom, a cyano group, a nitro group, a hydroxy group, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, and a substituted or unsubstituted C5-C30 condensed polycyclic group. Additionally, each of R₅, R₆and R₇ through R₁₀ may independently be, for example, a hydrogen atom, a halogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, a substituted or unsubstituted C5-C30 heteroaryloxy group, a substituted or unsubstituted C5-C30 condensed polycyclic group, an amino group, a substituted or unsubstituted C1-C30 alkylamino group, a substituted or unsubstituted C6-C30 arylamino group, a cyano group, a nitro group, a hydroxy group, a carboxyl group, a substituted or unsubstituted C1-C30 alkylcarboxyl group, a substituted or unsubstituted C6-C30 arylcarboxyl group, —SO₃H, a substituted or unsubstituted C1-C30 alkylsulfonyl group, and a substituted or unsubstituted C6-C30 arylsulfonyl group. The adjacent groups among R₅, R₆, and R₇ through R₁₀ may be capable of combining to form a saturated or unsaturated ring.

According to another aspect of the present invention, there is provided an organic EL display device that may include an organic film containing the above-described imidazole ring-containing compound positioned between a pair of electrodes.

### BRIEF DESCRIPTION OF THE DRAWINGS

FIG. 1 is a sectional view of a general organic electroluminescence (EL) display device.

FIG. 2 is the UV-vis spectrum and the photoluminescence (PL) spectrum of compound (I-1) according to an embodiment of the present invention.

FIG. 3 is the PL spectrum of a thin film formed using compound (I-1) according to an embodiment of the present invention.

FIG. 4 is a UV-vis spectrum and the photoluminescence (PL) spectrum of compound (I-4) according to an embodiment of the present invention.

FIG. 5 is the PL spectrum of a thin film formed using compound (I-4) according to an embodiment of the present invention.

FIG. 6 is the PL spectra of thin films formed using compounds (I-1) and (I-4) according to an embodiment of the present invention.

FIG. 7 is a graph of current density versus voltage for an organic EL display device manufactured in Example 3 according to an embodiment of the present invention.

FIG. 8 is a graph of luminance versus voltage for the organic EL display device manufactured in Example 3 according to an embodiment of the present invention.

FIG. 9 is a graph of efficiency versus luminance for the organic EL display device manufactured in Example 3 according to an embodiment of the present invention.

FIG. 10 is a graph of power efficiency versus luminance for the organic EL display device manufactured in Example 3 according to an embodiment of the present invention.

### DETAILED DESCRIPTION OF THE INVENTION

The present invention is directed to an imidazole ring-containing compound of Formula (1), as described above. Additionally, the compound of Formula (1) has strong blue luminescence and hole transporting characteristics and may be used as a blue luminescent material. Moreover, the compound may be used as a phosphorescent or a fluorescent host material.

In an embodiment of the present invention, R₁ and R₂, R₅ and R₆, or adjacent groups among R₇ through R₁₀ of Formula (1), may be capable of combining to form a saturated or unsaturated ring. This saturated or unsaturated ring may be a C6-C50 carbon ring or hetero ring.

According to an embodiment of the present invention, in Formula (2), as described above, each of R₁ and R₂ may be independently a C1-C12 alkyl group or a C6-C30 aryl group. Furthermore, the X of Formula (2) may be N, and R₃ may be a C2-C30 aryl group, for example.

According to a further embodiment of the present invention, R₁₁ may be a C6-C30 aryl group, and all of R₇ through R₁₀ may be hydrogen in Formula (3), as described above. In particular, in Formula (3) for example, X′may be O or S, R₄ may be a C6-C30 aryl group, and R₅ and R₆ may combine to form a C6-C30 saturated or unsaturated ring.

In a specific embodiment, examples of an imidazole ring-containing compound of Formula (1) include, but are not limited to, the compounds illustrated below.

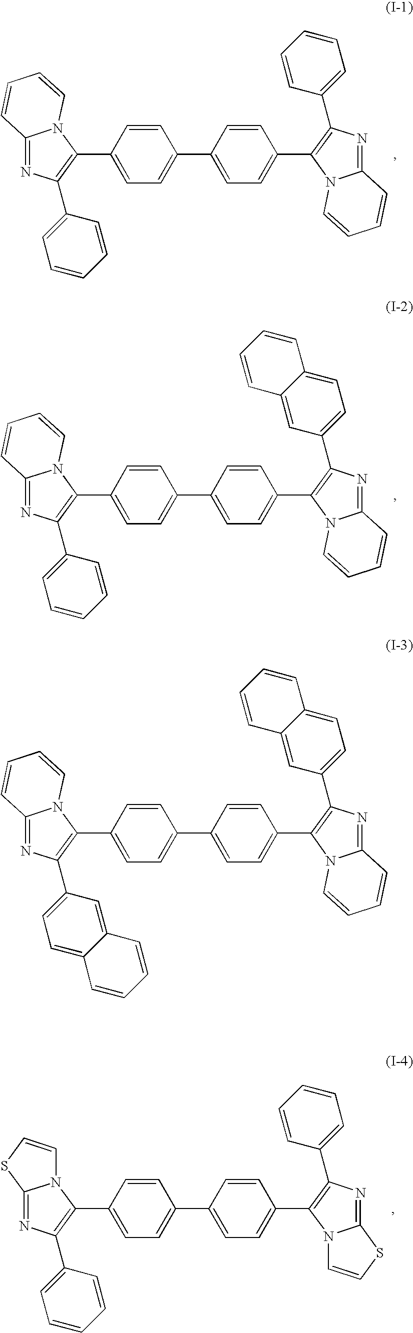

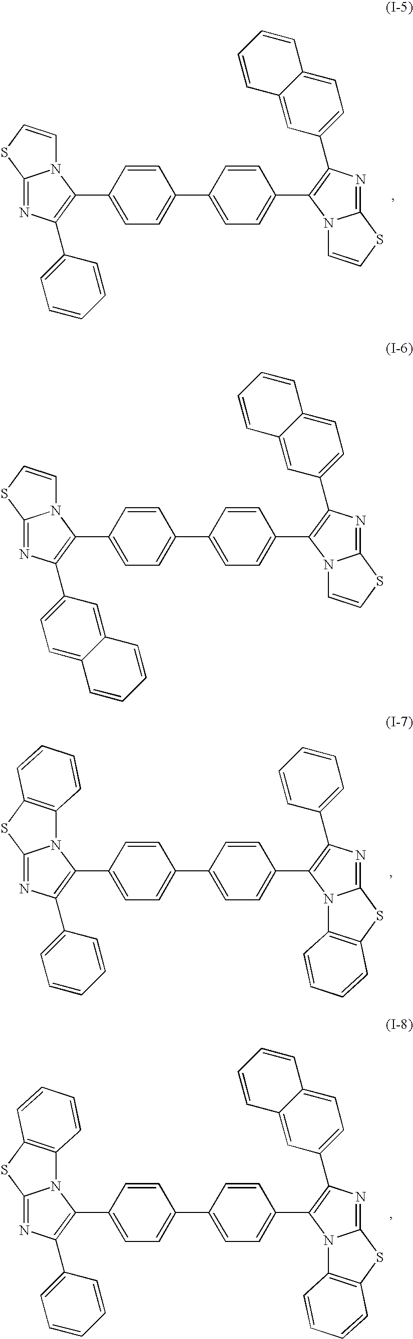

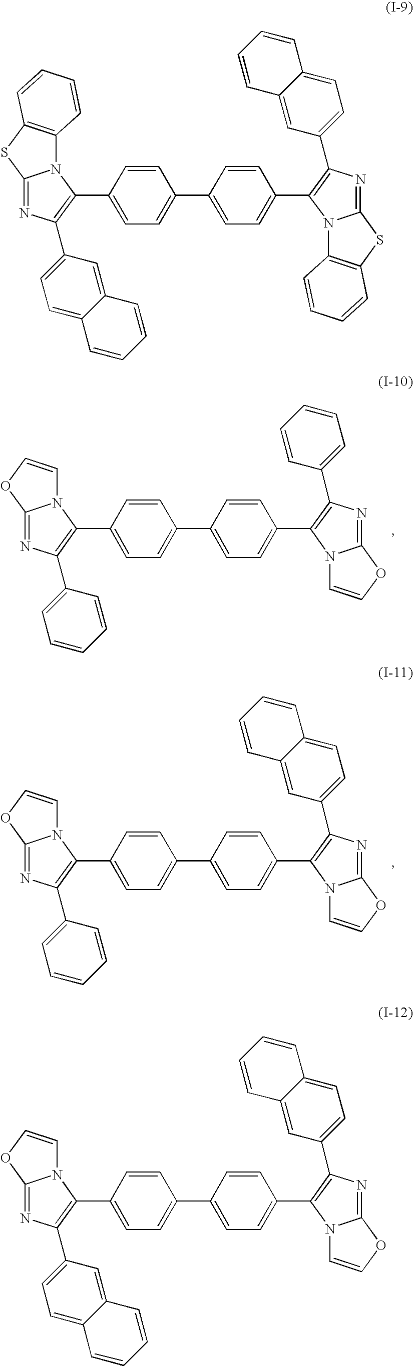

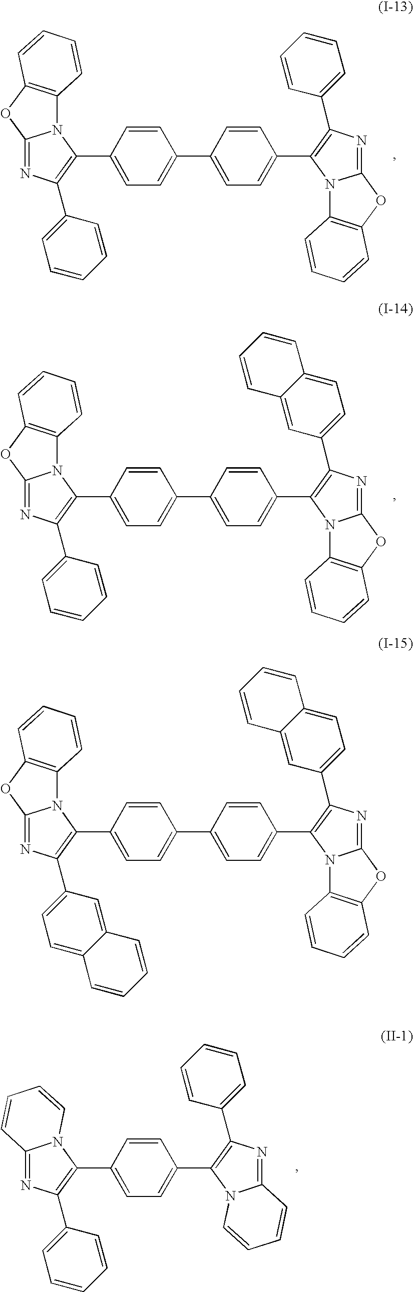

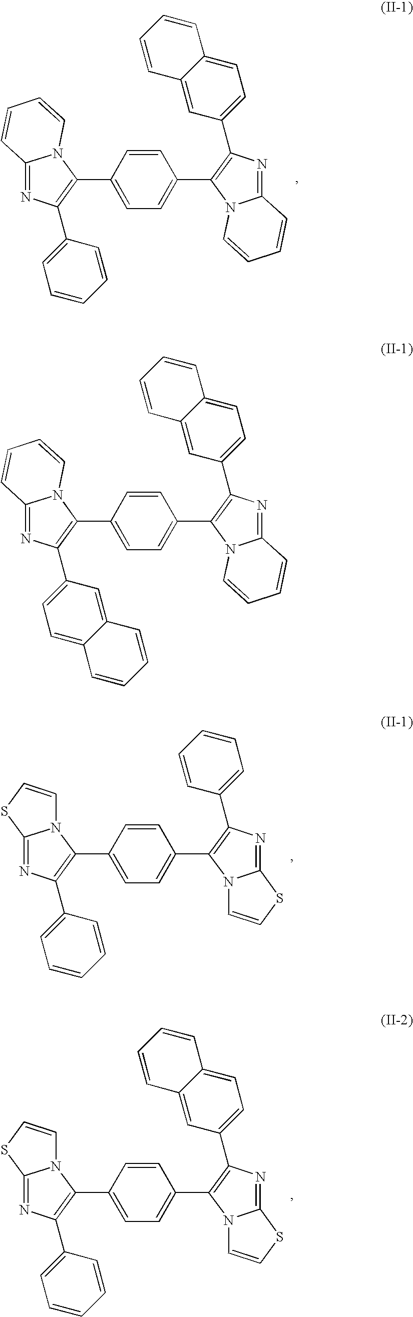

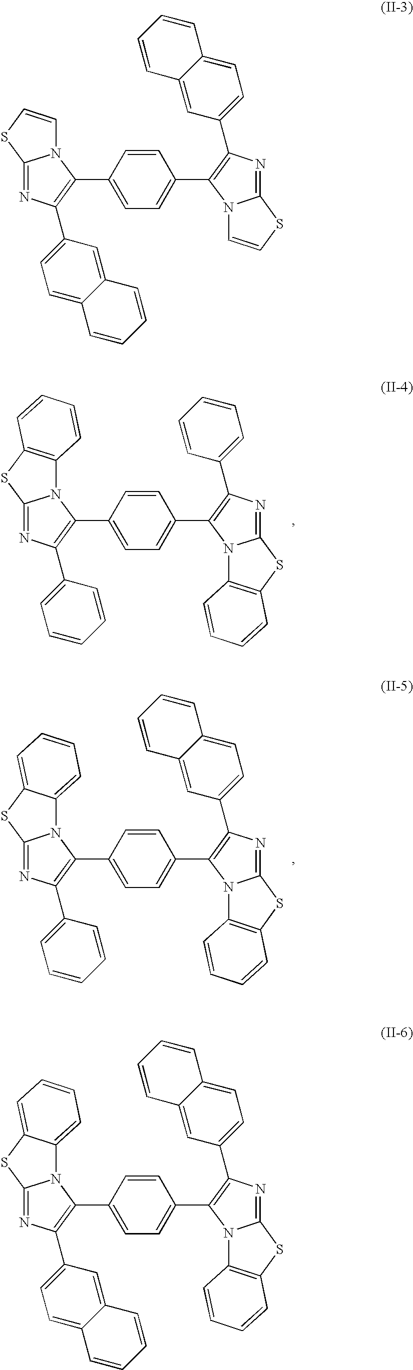

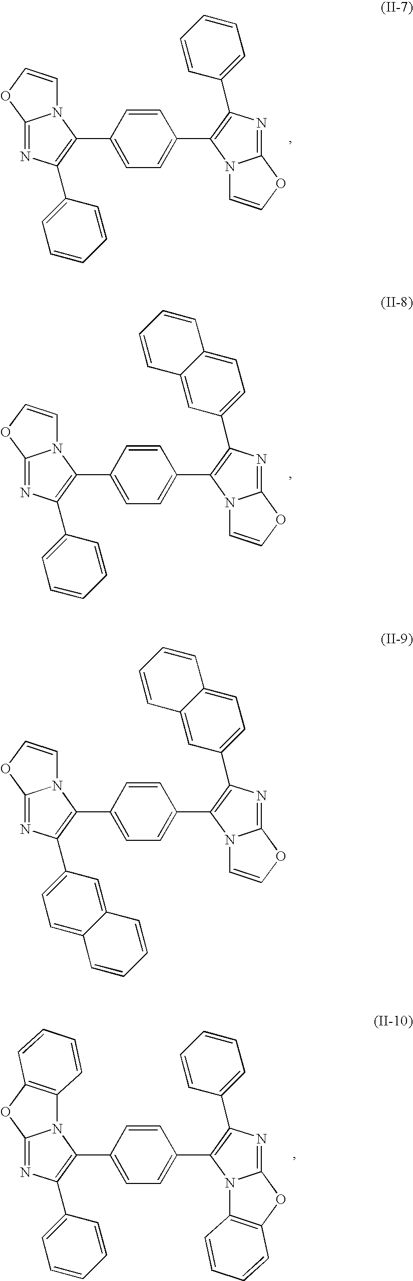

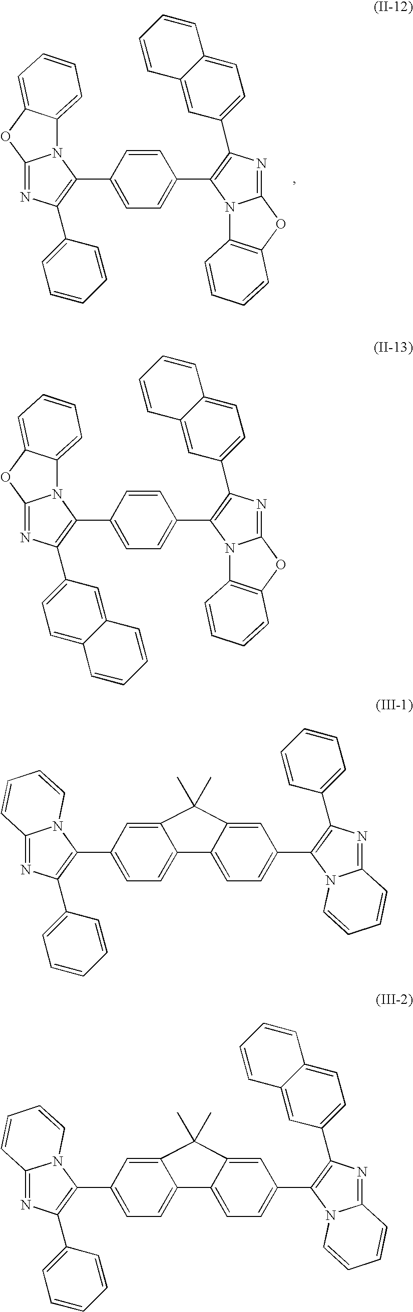

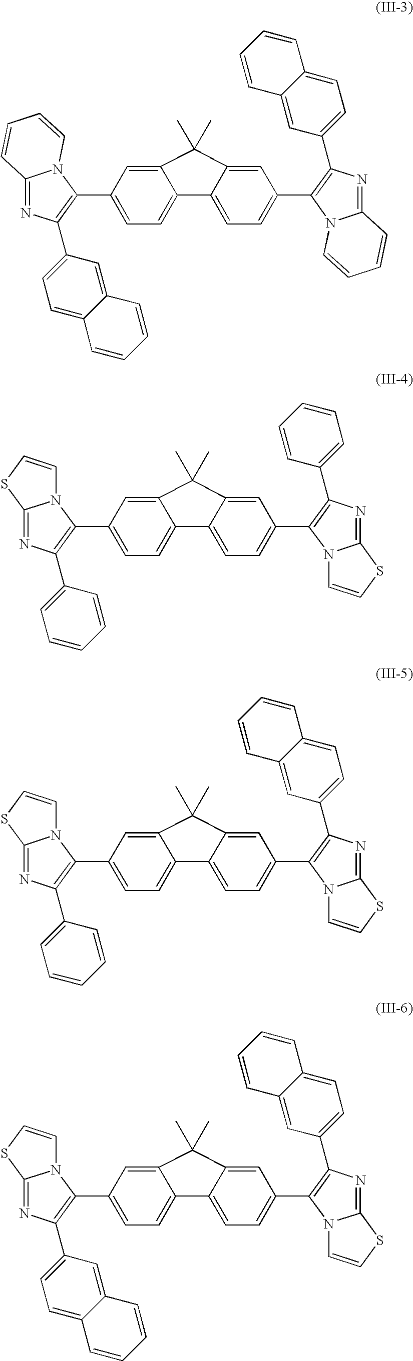

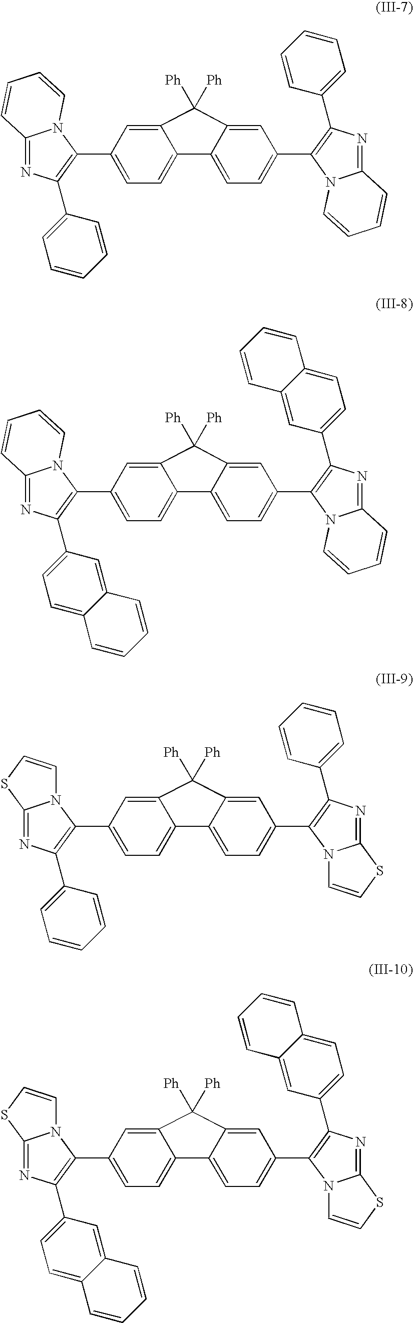

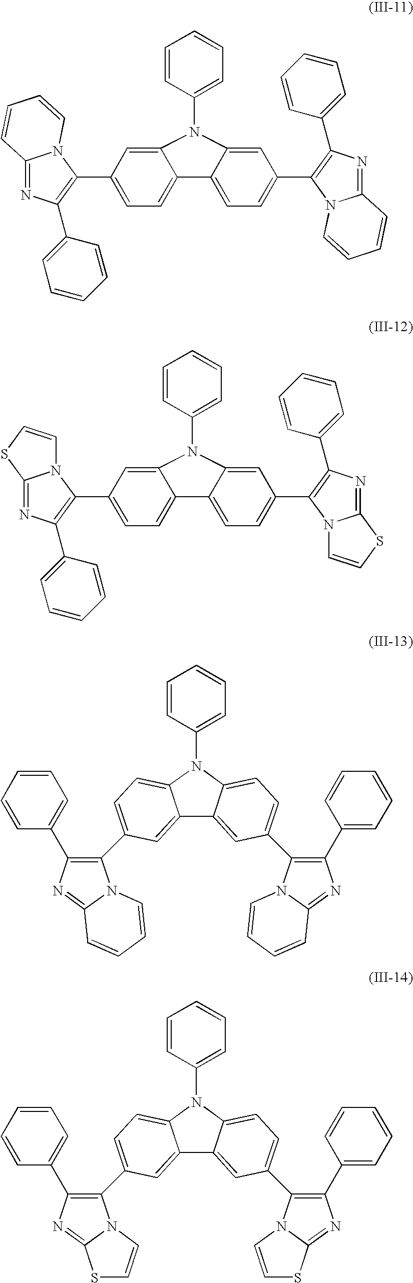

A method of manufacturing an organic EL display device using an organic film made of the imidazole ring-containing compound will be described below.

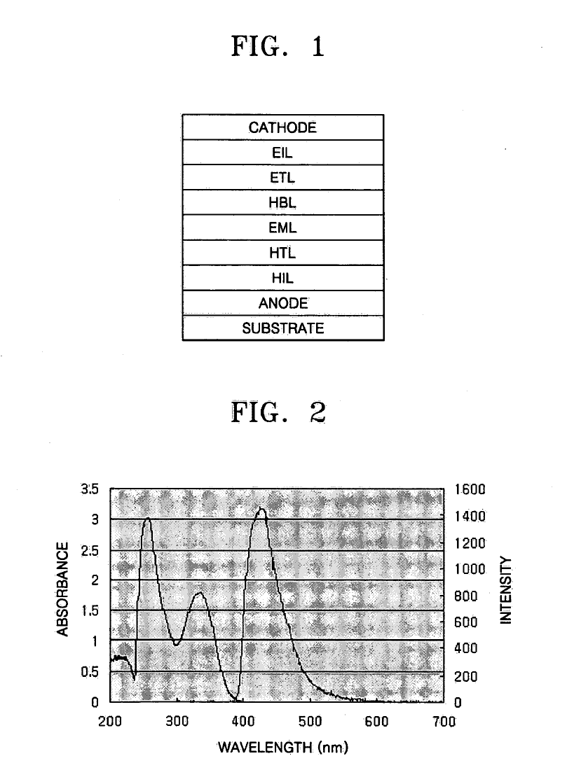

Referring to FIG. 1, which illustrates an embodiment of the present invention, a sectional view of a typical organic EL display device is shown. As shown in FIG. 1, an anode may be formed by coating an anode material on the surface of the substrate. Any substrate that is known in the art for organic EL devices may be used. Examples that may be used include a glass substrate and a transparent plastic substrate because they are easy to handle, waterproof and have an even surface. Examples of anode materials include, but are not limited to, indium tin oxide (ITO), indium zinc oxide (IZO), tin dioxide (SnO₂), zinc oxide (ZnO). These anode materials are transparent and have strong conductivity.

A hole injecting layer (HIL) may be formed by coating a HIL material on the anode using thermal vacuum deposition or spin coating. In particular, examples of HIL materials include, but are not limited to, CuPc, TCTA, m-MTDATA, and m-MTDAPB. These materials may be a Starbust type amine as illustrated below.

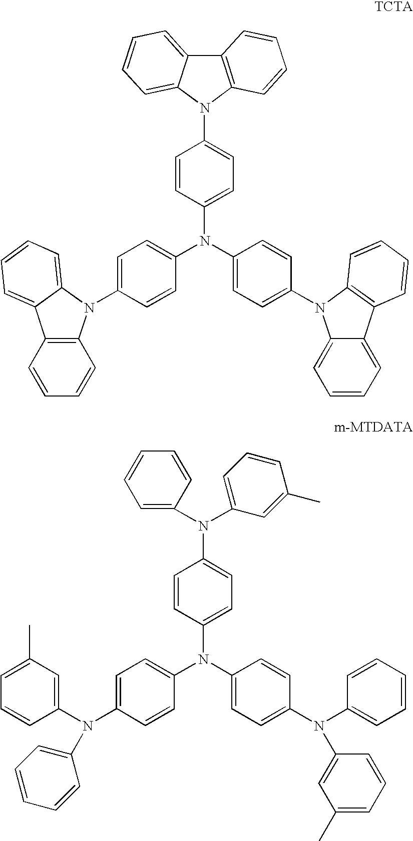

Additionally, a hole transporting layer (HTL) may be formed by coating a HTL material on the HIL using thermal vacuum deposition or spin coating. Examples of HIL materials, include, but are not limited to, N,N′-bis(3-methylphenyl)-N,N′-diphenyl-(1,1-biphenyl)-4,4′-diamine (TPD), and N,N′-di(naphthalene-1-yl)-N,N′-diphenyl benzidine(N,N′-di(naphthalene-1-yl)-N,N′-diphenyl-benzidine (α-NPB)).

An electroluminescent layer (EML) may be formed on the HTL. The EML may be made of any material, for example, the compound of Formula (1) may be used alone or in combination with a dopant. In the latter case when the compound of Formula (1) is used as the emissive host, fluorescent dopants that may be used together include IDE102 and IDE105 (Idemitsu Co., Southfield, Mich.), for example. Alternatively, phosphorescent dopants that may be used together include Ir(ppy)₃ (green), where “ppy” is an abbreviation for phenyipyridine, (4,6-F2ppy)₂Irpic (Adachi et al., 79 APPL. PHYS. LETT., 2082-2084 (2001)), and PtOEP (platinum(II)) octaethylporphyrin, for example. The EML may be fabricated using any method known in the art, such as thermal vacuum co-deposition, depending on the material used.

In a specific embodiment, the amount of a dopant used may be in a range of about 0.1 parts to about 20 parts by weight, and specifically, in the range of about 0.5 parts to about 12 parts by weight, with respect to 100 parts by weight of the EML material (the amount of the compound of Formula (1) used as a host plus the amount of the dopant). If the amount of the dopant is less than about 0.1 parts by weight, the effects of adding the dopant are trivial. If the amount of the dopant is greater than about 20 parts by weight, however, undesirable concentration quenching may occur in both phosphorescence and fluorescence.

An electron transporting layer (ETL) may be formed on the EML by vacuum deposition or spin coating, for example. A suitable material for the ETL includes, but is not limited to, Alq3. When the EML contains a phosphorescent dopant, a hole blocking layer (HBL) may be additionally formed on the EML by thermal vacuum deposition to prevent triplet excitons or holes from migrating into the ETL. Any material that is capable of transporting electrons and has a higher ionization potential than the emissive compound may be used for the HBL. In particular, the HBL materials may include, for example, Balq and BCP, as shown below.

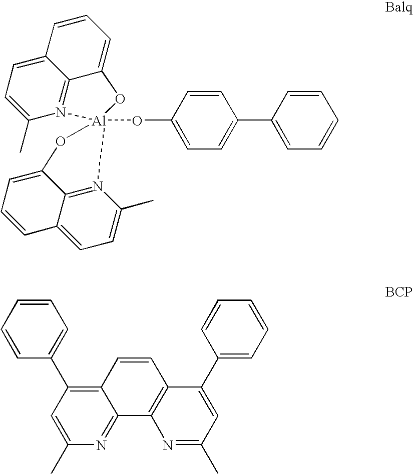

An electron injecting layer (EIL) may be optionally formed on the ETL. Examples of materials for the EIL may include, but are not limited to, LiF, NaCl, CsF, Li₂O, and BaO. Next, a cathode may be formed by coating a metal on the EIL by thermal vacuum deposition thereby completing the manufacture of the organic EL device. Suitable metals for the cathode may include Li, Mg, Al, Al-Li—Ca, Mg—In, and Mg—Ag, for example. A transmission type cathode may be formed using ITO and IZO to manufacture a front-emission device. An organic EL display device according to the present invention, which includes the anode, the HIL, the HTL, the EML, the HBL, the ETL, the EIL, and the cathode, may have an additional single or dual intermediate layer if required.

The present invention will be described in greater detail with reference to the following examples. The following examples are for illustrative purposes and are not intended to limit the scope of the invention.

### EXAMPLES

### Specific Example 1

### Synthesis of compound (VIII-2)

Synthesis of Intermediate (A)

1.99 g (10 mmol) of bromoacetophenone was dissolved in 50 ml of dimethoxyethane (DME), and 1 g (10 mmol) of 2-aminopyridine in a solid state was added to the solution, stirred for 5 hours at room temperature, and refluxed for 12 hours. The reaction product was distilled under reduced pressure to remove the solvent, and 60 ml of dichloromethane was added to dissolve the remaining product. The pH of the solution was adjusted to pH 10 using a 10% sodium carbonate solution. The dichloromethane phase was separated from the solution, and the remaining aqueous phase was extracted twice using 50 ml of dichloromethane. The collected organic phase was dried using magnesium sulfate and the solvent evaporated from the dried product. The resulting product was then purified by silica gel column chromatography to obtain 1.26 g of intermediate (A) with a yield of 65%. The structure of the compound was characterized using proton NMR as follows: ¹H NMR (CDCl₃, 300 MHz) δ (ppm) 8.1 (d, 1H), 8.03-7.90 (m, 2H), 7.80 (d, 1H), 7.60 (dd, 1H), 7.51-7.40 (m, 2H), 7.39-7.27 (m, 1H), 7.21-7.08 (m, 1H), 1.43 (dd, 1H); ¹³C NMR (CDCl₃, 100 MHz) δ (ppm) 145.7, 145.7 133.7, 128.7, 128.6, 127.9, 126.0, 124.5, 117.4, 112.3, 108.1.

Synthesis of Intermediate (B)

400 mg (2 mmol) of intermediate A was dissolved in 10 ml of pyridine, and 760 mg (3 mmol) of iodine was added and stirred for 5 hours at 50° C. A saturated oxalic acid solution was added to stop the reaction, and an extraction was performed three times using 10 ml of dichloromethane. The collected organic phase was dried using magnesium sulfate and the solvent evaporated from the dried product. The resulting product was then purified by silica gel column chromatography to obtain 462 mg of intermediate (B) with a yield of 72%. The structure of the compound was analyzed using proton NMR as follows: ¹H NMR (CDCl₃, 300 MHz) δ (ppm) 8.2 (d, 1H), 8.12-8.02 (m,2H), 7.60 (d, 1H), 7.54-7.44 (m,2H), 7.43-7.34 (m,1H), 7.28-7.19 (m,1H), 6.91 (d, 1H).

Synthesis of Intermediate (C)

6 g (50 mmol) of bromoacetophenone was dissolved in 250 ml of DME, and 10 g (50 mmol) of 2-aminothiazole in a solid state was added to the solution, stirred for 5 hours at room temperature, and refluxed for 12 hours. The reaction product was distilled under reduced pressure to remove the solvent, and 250 ml of dichloromethane was added to dissolve the remaining product. The pH of the solution was adjusted to pH 10 using a 10% sodium carbonate solution. The dichloromethane phase was separated from the solution, and the remaining aqueous phase was extracted twice using 200 ml of dichloromethane. The collected organic phase was dried using magnesium sulfate and the solvent evaporated from the dried product. The resulting product was then purified by silica gel column chromatography to obtain 8.4 g of intermediate (C) with a yield of 84%.

Synthesis of Intermediate (D)

1 g (5 mmol) of intermediate (C) was dissolved in 15 ml of pyridine, and 1.9 g (7.5 mmol) of iodine was added and stirred for 5 hours at 50° C. A saturated oxalic acid solution was added to stop the reaction, and an extraction was performed three times using 20 ml of dichloromethane. The collected organic phase was dried using magnesium sulfate and the solvent evaporated from the dried product. The resulting product was then purified by silica gel column chromatography to obtain 1.1 g of intermediate (D) with a yield of 73%. The compound was characterized using proton NMR as follows: ¹H NMR (CDCl₃, 400 MHz) δ (ppm) 8.00 (d, 2H), 7.47-7.42 (m, 7H), 7.37-7.32 (m, 1H), 6.90 (s, 1H).

Synthesis of Intermediate (E)

4.5 g (14.4 mmol) of 4,4′-dibromobiphenyl was dissolved in 40 ml of THF and 15 ml (36 mmol) of 2.5M n-butyl lithium dissolved in n-hexane was added dropwise at 78° C. and stirred for 2 hours. 8.1 ml (72 mmol) of trimethyl borate was added to the reaction solution and stirred for 3 hours at the same temperature and further for 12 hours at room temperature. The pH was adjusted to pH 1 by using 12M hydrochloric acid, and the solution was stirred for 2 hours at room temperature. Then, the pH was adjusted to pH 14 using an aqueous solution of 4M NaOH, and an extraction was performed three times using 50 ml of diethyl ether each time. The collected organic phase was dried using magnesium sulfate and the solvent evaporated from the dried product. The resulting product was then purified by silica gel column chromatography to obtain 1.7 g of intermediate (E) in white solid form with a yield of 49%.

Synthesis of Compound (I-1)

2.4 g (7.5 mmol) of intermediate (B) and 605 mg (2.5 mmol) of intermediate (E) were dissolved in 20 ml of THF, and 115 mg (0.1 mmol) of tetrakistriphenylphosphinepalladium and a solution of 3.5 mg (25 mmol) of K₂CO₃ in 15 ml of distilled water were added sequentially and stirred at 75° C. for 12 hours. After the reaction was completed, the reaction solution was extracted three times using 30 ml of ethyl acetate each time. The collected organic phase was dried using magnesium sulfate and the solvent evaporated from the dried product. The resulting product was then purified by silica gel column chromatography to obtain 1 g of compound (I-1) with a yield of 72%. This compound was sublimated and purified at 320° C. in a 1-torr nitrogen atmosphere using a sublimating and purifying apparatus to obtain a white solid compound. The structure of this compound was identified by ¹H NMR as follows: ¹H NMR (CDCl₃, 400 MHz) δ (ppm) 8.07 (d, 2H), 7.86 (d, 4H), 7.72 (dd, 6H), 7.59 (d, 4H), 7.34-7.21 (m, 8H), 6.79 (dd, 2H); ¹³C NMR (CDCl₃, 100 MHz) δ (ppm) 144.8, 142.3, 140.4, 133.6, 131.1, 128.8, 128.2, 128.1, 127.9, 127.6, 125.3, 123.2, 120.6, 117.0, 112.6.

Compound (I-1) obtained in Specific Example 1 was diluted to 0.2 mM using CHCl₃ for UV spectrum measurement. The results indicated that the compound (I-1) has a maximum absorption peak at 333 nm. Compound (I-1) was diluted to 10 mM using CHCl₃ to measure its PL characteristics. The results showed a maximum emission peak appearing at 425 nm (FIG. 2) for the compound. The color purity of the compound at this wavelength was CIE(x,y):0.1606, 0.0581 in an NTSC chromaticity coordinate system.

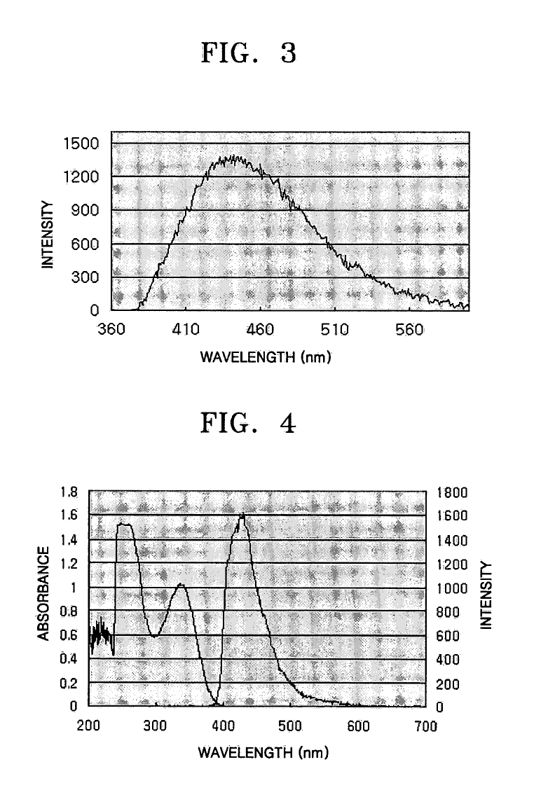

A solution obtained by dissolving compound (I-1) and polymethylmethacrylate (PMMA) in a ratio of 15:1 by weight was spin-coated on a glass substrate (1.0T, 50 mm×50 mm) to form a thin film. The PL characteristics of the film were measured. The results showed a maximum emission peak appearing at 442 nm (FIG. 3) for the compound. The color purity of the film at this wavelength was CIE(x,y):0.1633, 0.1598 in an NTSC chromaticity coordinate system.

The compound was analyzed using an AC-2, which is a UV absorption spectrum and ionization potential measuring system. The analysis indicated the HOMO (Highest Occupied Molecular Orbital) energy level to be 5.81 eV, and the LOMO (Lowest Occupied Molecular Orbital) energy level to be 2.65 eV.

### Specific Example 2:

### Synthesis of Compound (I-4)

2.45 g (7.5 mmol) of intermediate (D) and 605 mg (2.5 mmol) of intermediate (E) were dissolved in 20 ml of THF, and 115 mg (0.1 mmol) of tetrakistriphenylphosphinepalladium and a solution of 3.5 mg (25 mmol) of K₂CO₃ in 15 ml of distilled water were added sequentially and stirred at 75° C. for 12 hours. After the reaction was completed, the reaction solution was extracted three times using 30 ml of ethyl acetate each time. The collected organic phase was dried using magnesium sulfate and the solvent evaporated from the dried product. The resulting product was then purified by silica gel column chromatography to obtain 1 g of compound (I-4) with a yield of 77%. The structure of this compound was identified by ¹H NMR as follows: ¹H NMR (CDCl₃, 400 MHz) δ (ppm) 7.75 (d, 4H), 7.66 (dd, 4H), 7.55 (d, 4H) 7.45 (d, 2H), 7.33-7.24 (m, 6H), 6.85 (d, 2H).

Compound (I-4) obtained in Specific Example 2 was diluted to 0.2 mM using CHCl₃ for UV spectrum measurement. The results indicated that the compound (I-4) has a maximum absorption peak at 336 nm. Compound (I-4) was diluted to 10 mM using CHCl₃ to measure its PL characteristics at 336 nm. The results showed a maximum emission peak appearing at 430 nm (FIG. 4) for the compound. The color purity of the compound at this wavelength was CIE(x,y):0.1645, 0.0671 in an NTSC chromaticity coordinate system.

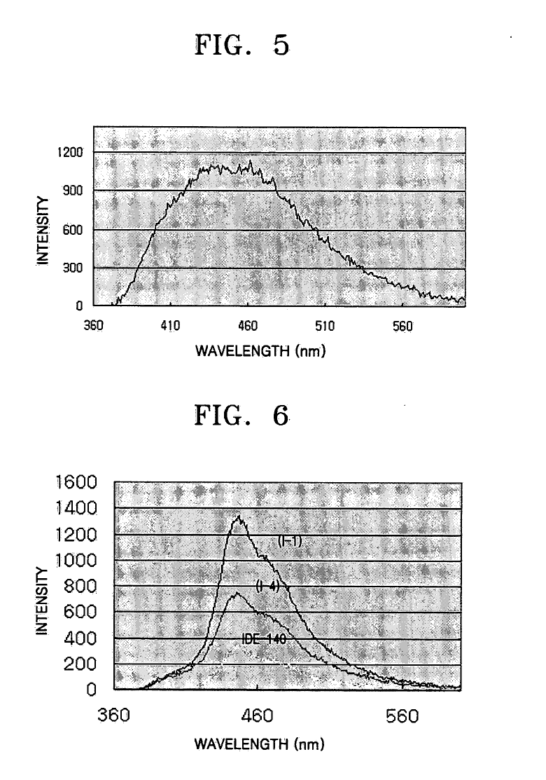

A solution obtained by dissolving compound (I-4) and PMMA in a ratio of 15:1 by weight was spin-coated on a glass substrate (1.0T, 50 mm ×50 mm) to form a thin film. The PL characteristics of the film were measured. The analysis showed a maximum emission peak appearing at 443 nm (FIG. 5) for the compound. The color purity of the film at this wavelength was CIE(x,y):0.1794, 0.1828 in an NTSC chromaticity coordinate system.

A thin film was coated using a mixture of 95 parts by weight of compound (I-1) as a fluorescent host and 5 parts by weight of IDE 105 (Idemitsu Co., Southfield, Mich.) as the blue fluorescent dopant. A thin film was coated using a mixture of 95 parts by weight of compound (I-4) as a fluorescent host and 5 parts by weight of IDE 105 (Idemitsu Co.) as the blue fluorescent dopant. The PL characteristics of each of the thin films was measured and compared with the PL characteristics of the blue fluorescent host IDE140 (Idemitsu Co.) (FIG. 6). The results indicated that both compounds, (I-1) and (I-3), have much greater maximum absorption peaks at 444 nm than IDE140.

The compound was analyzed using an AC-2, which is a UV absorption spectrum and ionization potential measuring system. The analysis showed the HOMO (Highest Occupied Molecular Orbital) energy level of the film containing compound (I-4) to be 5.76 eV, and the LOMO (Lowest Occupied Molecular Orbital) energy level to be 2.66 eV.

### Specific Example 3

### Manufacture of Organic EL Display Device

An indium tin oxide (ITO) glass substrate (Coming Co., Coming, N.Y.) having a resistance of 15Ω/ cm² (1200 Å) was cut to a size of 50 mm×50 mm×0.7 mm, cleaned by ultrasonication in isopropyl alcohol for 5 minutes and then in pure water for 5 minutes. Additionally, the substrate was further cleaned for 30 minutes by UV ozone cleaning, and then used as an anode.

A hole injecting layer was formed of IDE 406 (Idemitsu Co.) on the anode to a thickness of 600 Å by vacuum deposition. Next, a hole transporting layer was formed using 4,4′-bis[N-(1-naphthyl)-N-phenylamino]biphenyl (NPB) on the hole injecting layer by vacuum deposition to a thickness of 300 Å.

An electroluminescent layer was formed of compound (I-1) on the hole transporting layer to a thickness of 200 Å by vacuum deposition. Next, an electron transporting layer was formed of Alq3 on the electroluminescent layer to a thickness of 250Å by vacuum deposition. LiF and Al were sequentially deposited on the electron transporting layer to a thickness of 10 Å and 3000 Å, respectively, by vacuum deposition to form an electron injecting layer and an anode, thereby resulting in a complete organic EL display device.

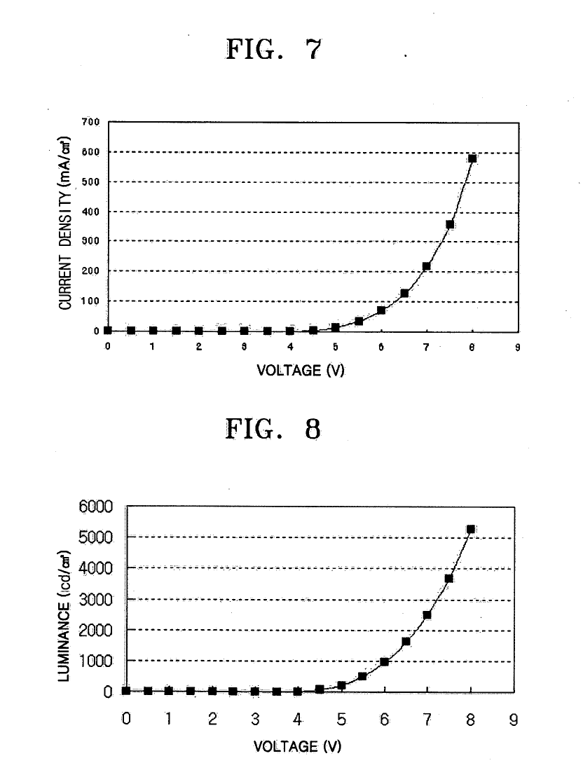

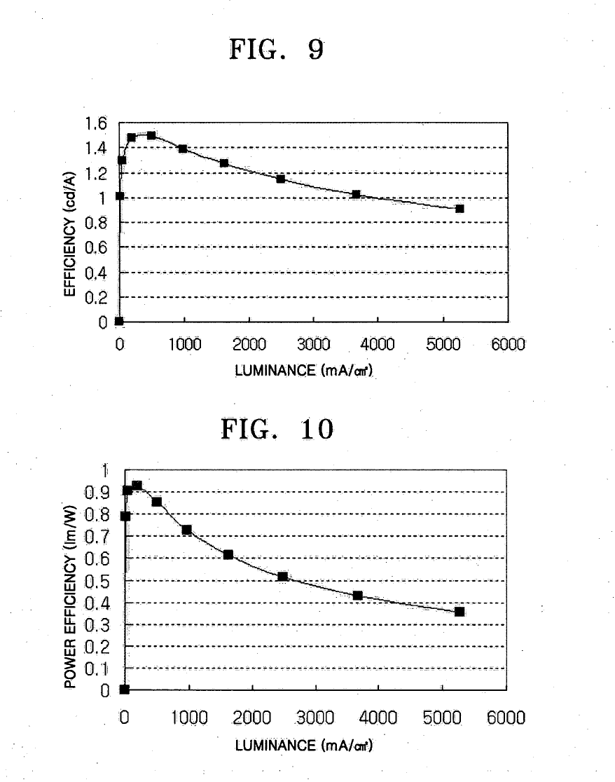

The luminance, luminescent efficiency, and color coordinate characteristics of the organic EL display device manufactured in Example 3 were measured. The results illustrated in FIGS. 7-10, show that the organic EL display device has a luminance of 501 cd/M² and a luminescent efficiency of 1.49 cd/A at a DV voltage of 5.5V and a color coordinate CIE(x,y): (0.168, 0.178), indicating that the device can emit high purity, strong blue light.

As described above, an imidazole ring-containing compound of Formula (1) according to the present invention has strong blue luminescence and hole transporting characteristics and may be used as a blue luminescent material and as a host of phosphorescent and fluorescent dopants in various colors such as red, green, blue, and white. In addition, an organic EL display device manufactured using the imidazole ring-containing compound has high-efficiency luminescence characteristics and consumes less power.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. An imidazole ring-containing compound, comprising:
Formula 1
Ar₁-Ar₂-Ar₃
where Ar₂ is selected from the group consisting of groups of formula (2): 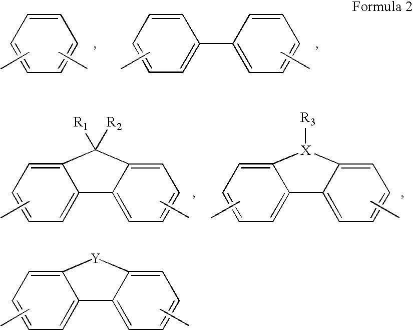
where X is selected from the group consisting of N, B, and P; Y is selected from the group consisting of O, S, and Se; and each of R₁, R₂, and R₃ is independently selected from the group consisting of a hydrogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C2-C30 heteroacryl group, a substituted or unsubstituted C5-C30 condensed polycyclic group, heteroaryloxy group, a substituted or unsubstituted C6-C30 condensed polycyclic group, and R₁ and R₂ can combine together to form a saturated or unsaturated ring; and
where each of Ar₁ and Ar₃ is independently selected from the group consisting of groups of Formula (3): 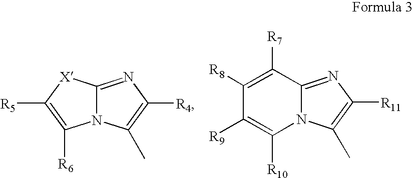
where X′ is selected from the group consisting of O, S, and Se;
where each of R₄ and R₁₁ is independently selected from the group consisting of a hydrogen atom, a halogen atom, a cyano group, a nitro group, a hydroxy group, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, and a substituted or unsubstituted C5-C30 condensed polycyclic group; and
where each of R₅, R₆, and R₇ through R₁₀ is independently selected from the group consisting of a hydrogen atom, a halogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, a substituted or unsubstituted C5-C30 heteroaryloxy group, a substituted or unsubstituted C5-C30 condensed polycyclic group, an amino group, a substituted or unsubstituted C1-C30 alkylamino group, a substituted or unsubstituted C6-C30 arylamino group, a cyano group, a nitro group, a hydroxy group, a carboxyl group, a substituted or unsubstituted C1-C30 alkylcarboxyl group, a substituted or unsubstituted C6-C30 arylcarboxyl group, —SO₃H, a substituted or unsubstituted C1 -C30 alkylsulfonyl group, and a substituted or unsubstituted C6-C30 arylsulfonyl group, and where adjacent groups among R₅, R₆, and R₇ through R₁₀ can combine together to form a saturated or unsaturated ring.

2. The imidazole ring-containing compound of claim 1, wherein each of R₁ and R₂ in said Formula (2) is independently a C1-C12 alkyl group or a C6-C30 aryl group.

3. The imidazole ring-containing compound of claim 1, wherein said X in said Formula (2), is said N, and R₃ is said C6-C30 aryl group.

4. The imidazole ring-containing compound of claim 1, wherein, in said Formula (3), R₁₁ is a C6-C30 aryl group, and all of R₇ through R₁₀ are hydrogen.

5. The imidazole ring-containing compound of claim 1, wherein, in said Formula (3), X′ is O or S, R₄ is a C6-C30 aryl group, and R₅ and R₆ combine together to form a C6-C30 saturated or unsaturated ring.

6. The imidazole ring-containing compound of claim 1, wherein said imidazole-ring containing compound is a compound selected from the group consisting of:

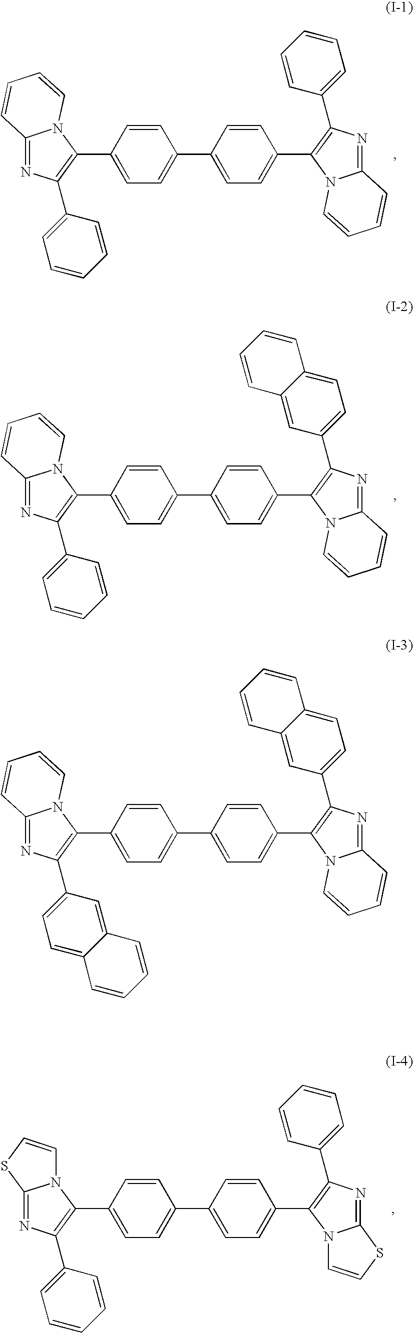

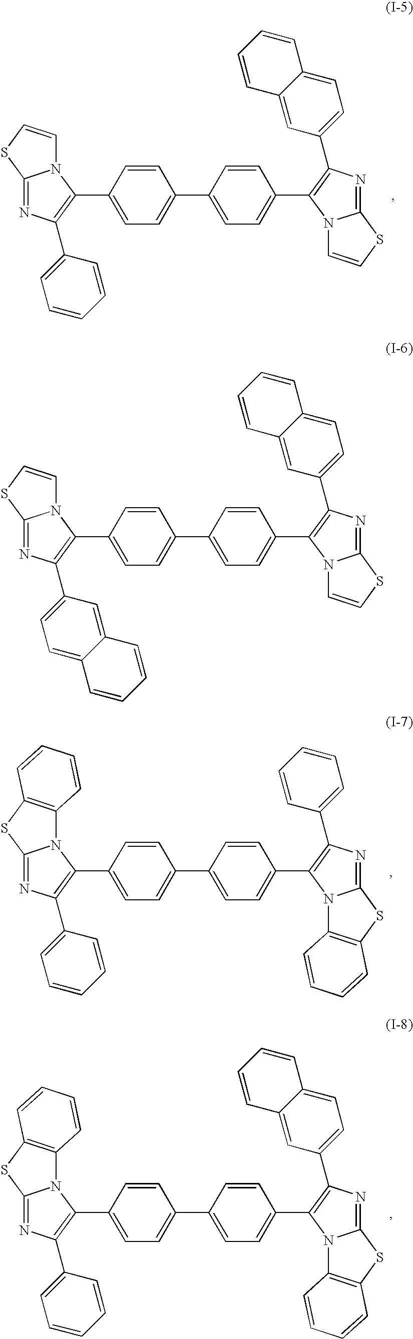

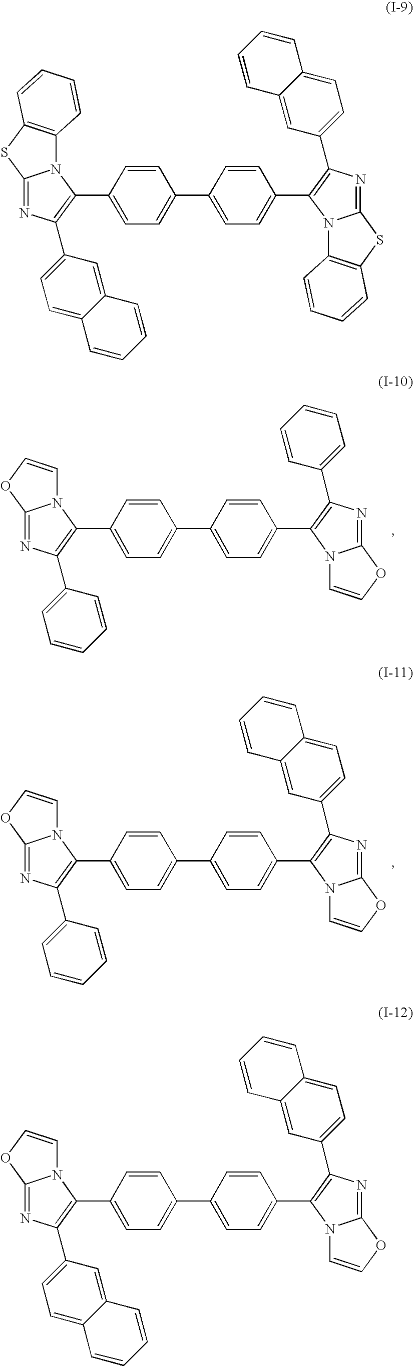

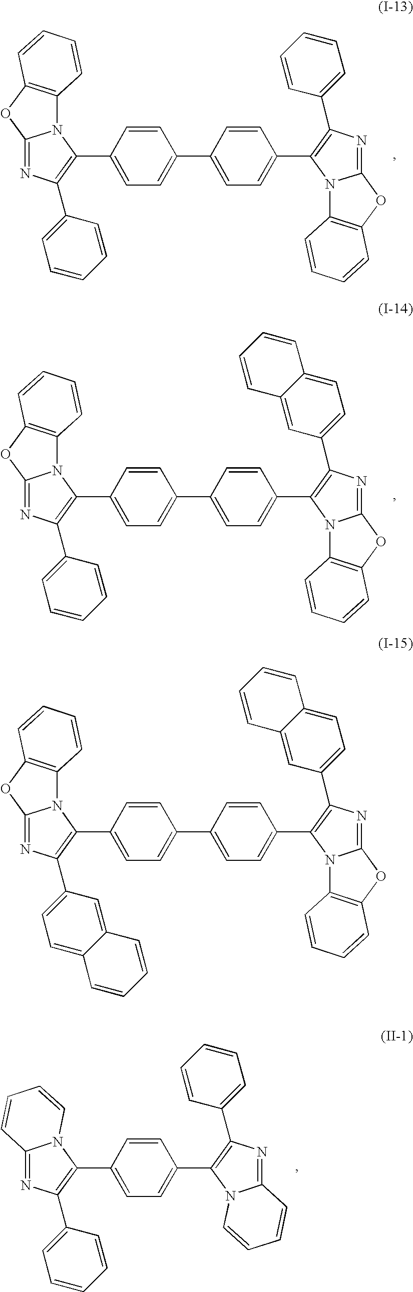

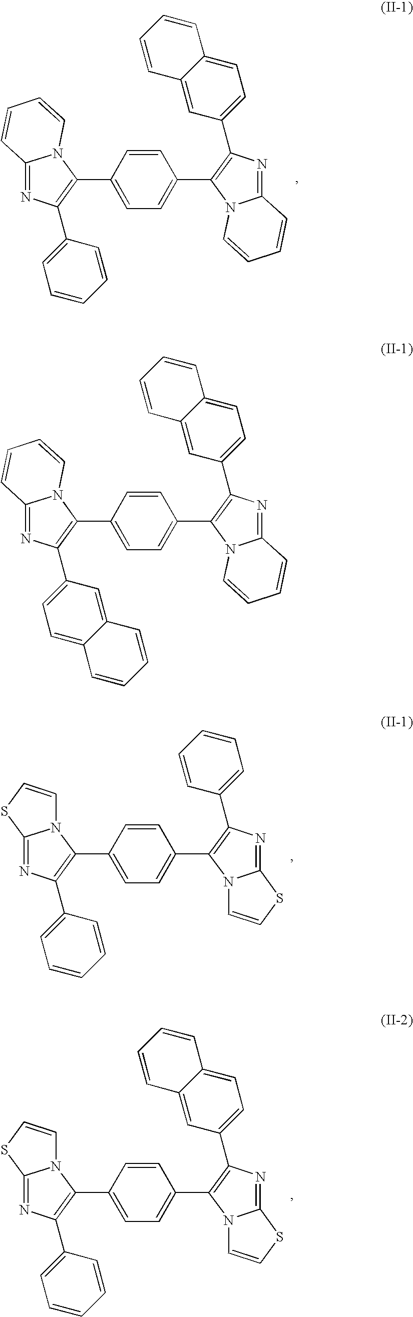

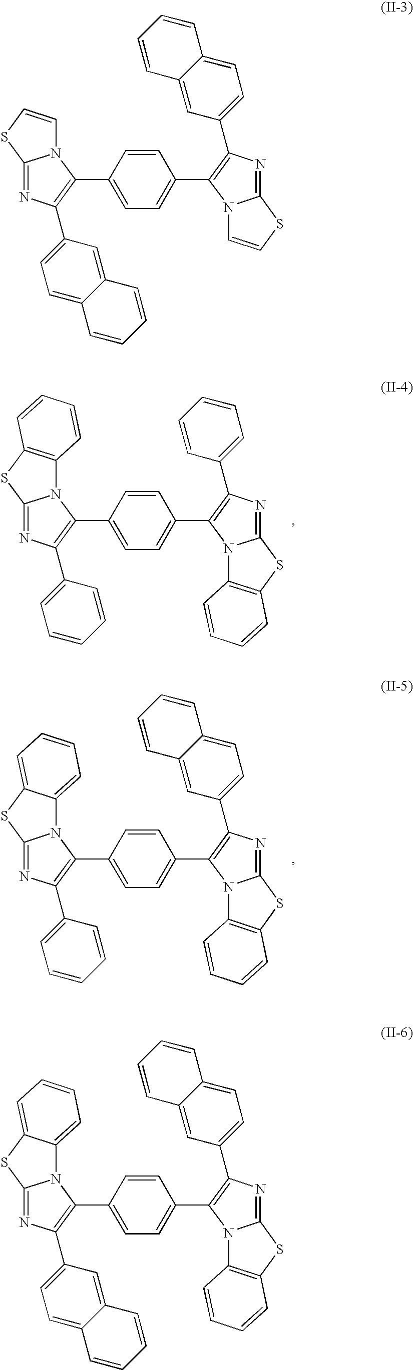

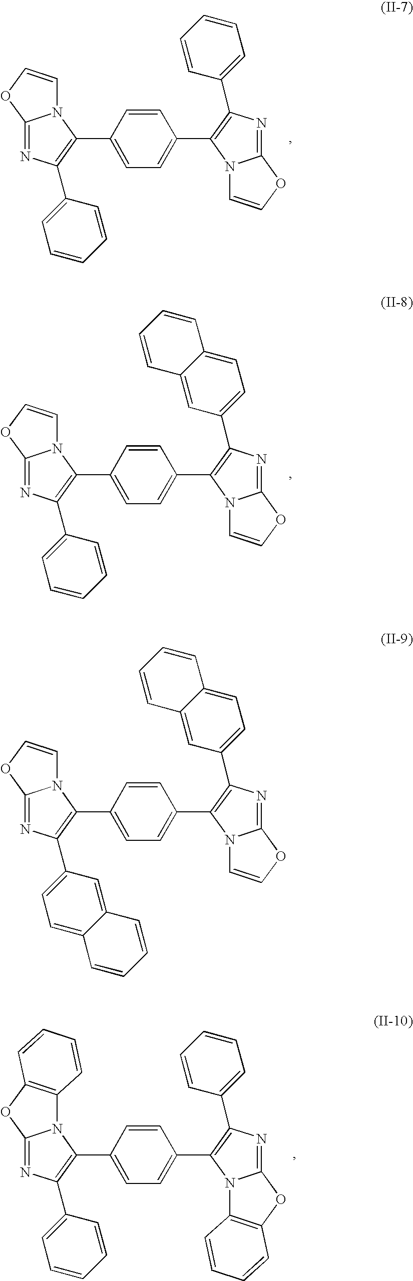

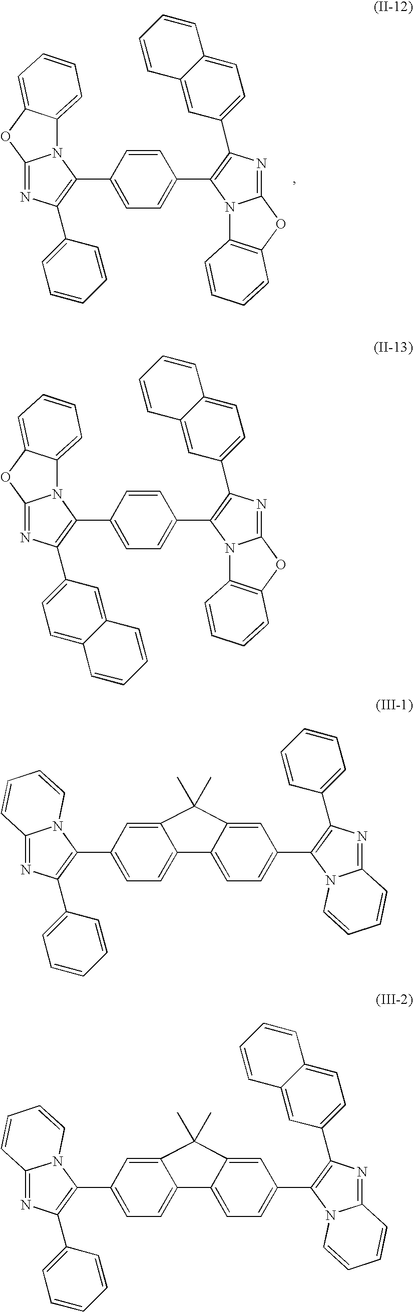

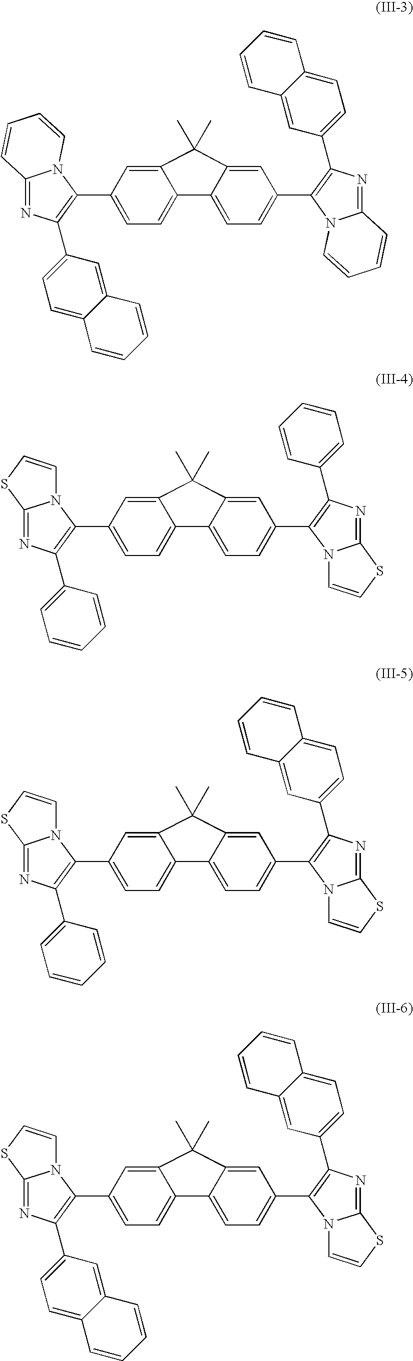

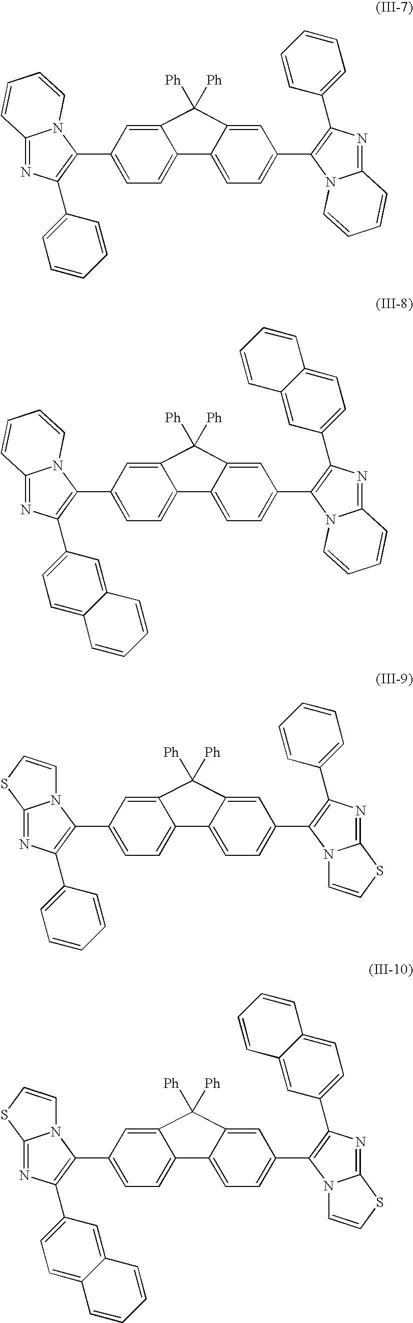

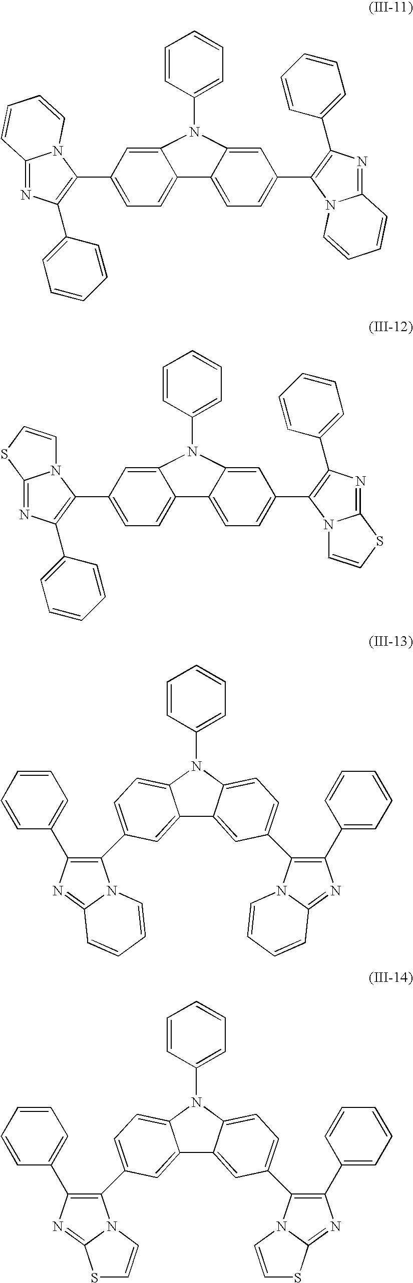

7. An organic electroluminescence display device comprising:
an organic film between a pair of electrodes, wherein the organic film comprises an imidazole ring-containing compound of Formula (1):
Formula 1
Ar₁-Ar₂-Ar₃
where Ar₂ is group selected from the group consisting of: 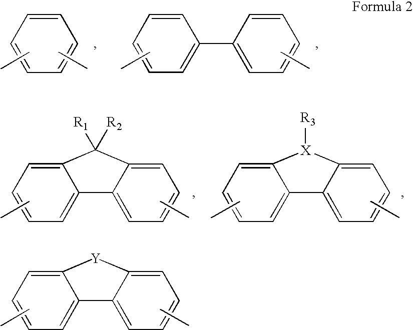
where X is selected from the group consisting of N, B, and P; Y is selected from the group consisting of O, S, and Se; and where each of R₁, R₂, and R₃ is independently selected from the group consisting of a hydrogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C2-C30 heteroacryl group, a substituted or unsubstituted C5-C30 condensed polycyclic group, a heteroaryloxy group, a substituted or unsubstituted C6-C30 condensed polycyclic group, and R₁ and R₂ can combine together to form a saturated or unsaturated ring; and
where each of Ar₁ and Ar₃ is independently selected from the group consisting of: 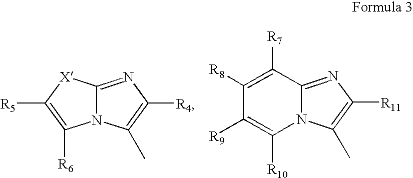
where X′ is selected from the group consisting of O, S, and Se;
where each of R₄ and R₁₁ is independently selected from the group consisting of a hydrogen atom, a halogen atom, a cyano group, a nitro group, a hydroxy group, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, and a substituted or unsubstituted C5-C30 condensed polycyclic group; and
where each of R₅, R6, and R₇ through R₁₀ is independently selected from the group consisting of a hydrogen atom, a halogen atom, a substituted or unsubstituted C1-C30 alkyl group, a substituted or unsubstituted C1-C30 alkoxy group, a substituted or unsubstituted C6-C30 aryl group, a substituted or unsubstituted C6-C30 aryloxy group, a substituted or unsubstituted C5-C30 heteroacryl group, a substituted or unsubstituted C5-C30 heteroaryloxy group, a substituted or unsubstituted C5-C30 condensed polycyclic group, an amino group, a substituted or unsubstituted C1-C30 alkylamino group, a substituted or unsubstituted C6-C30 arylamino group, a cyano group, a nitro group, a hydroxy group, a carboxyl group, a substituted or unsubstituted C1-C30 alkylcarboxyl group, a substituted or unsubstituted C6-C30 arylcarboxyl group, -SO₃H, a substituted or unsubstituted C1-C30 alkylsulfonyl group, and a substituted or unsubstituted C6-C30 arylsulfonyl group, and where adjacent groups among R₅, R₆, and R₇ through R₁₀ can combine together to form a saturated or unsaturated ring.

8. The organic electroluminescence display device of claim 7, wherein the organic film is an electroluminescent layer.

9. The organic electroluminescence display device of claim 8, wherein the electroluminescent layer further comprises a phosphorescent or fluorescent dopant emitting visible range light.

10. The organic electroluminescence display device of claim 7, wherein the organic film is a hole injecting layer or a hole transporting layer.

11. The organic electroluminescence display device of claim 7, wherein each of R₁ and R₂ in said Formula (2) is independently a C1-C12 alkyl group or a C6-C30 aryl group.

12. The organic electroluminescence display device of claim 7, wherein said Formula (2), X is N, and R₃ is a C6-C30 aryl group.

13. The organic electroluminescence display device of claim 7, wherein said Formula (3), R₁₁ is a C6-C30 aryl group, and all of R₇ through R₁₀ are hydrogen.

14. The organic electroluminescence display device of claim 7, wherein said Formula (3), X′ is O or S, R₄ is a C6-C30 aryl group, and R₅ and R₆ combine together to form a C6-C30 saturated or unsaturated ring.

15. The organic electroluminescence display device of claim 7, wherein the imidazole ring-containing compound is a compound selected from the group consisting of: 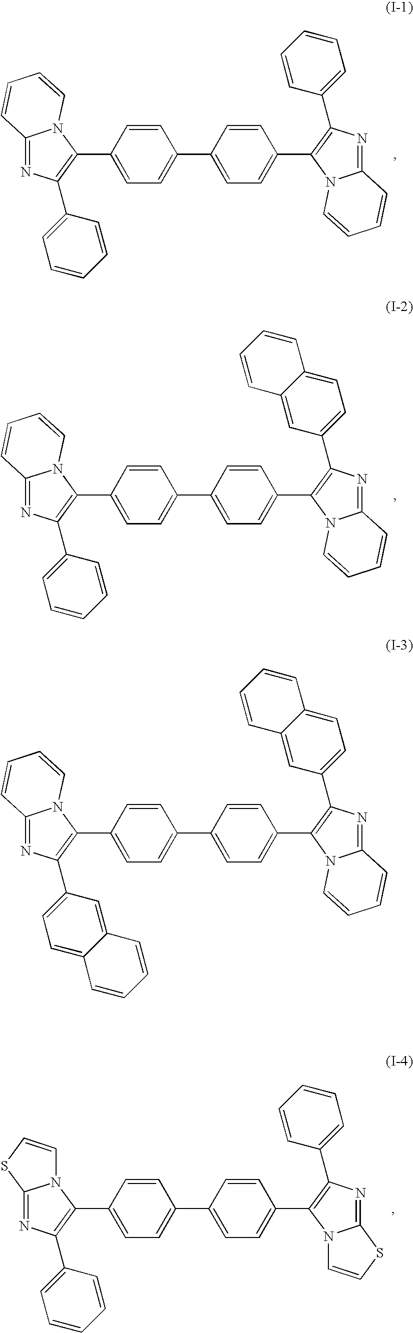 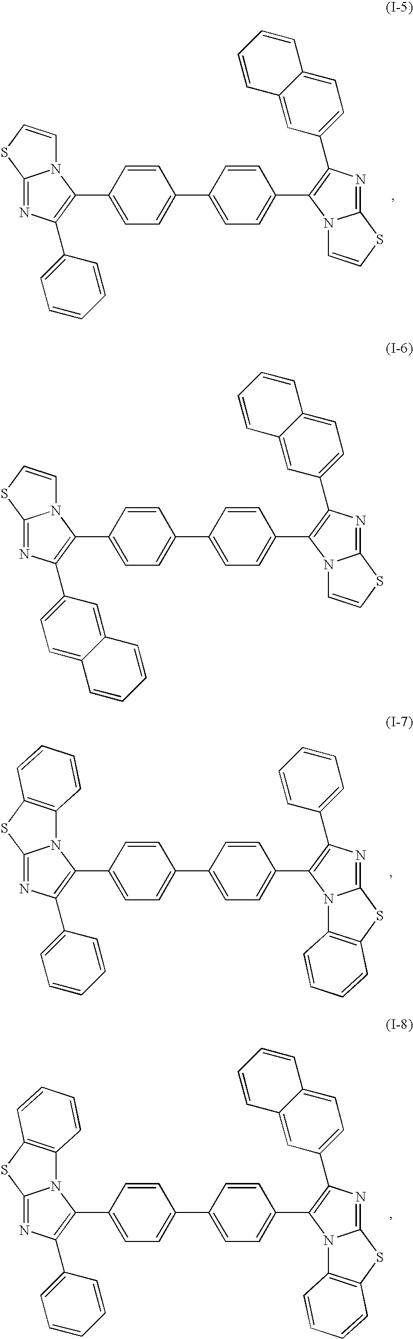 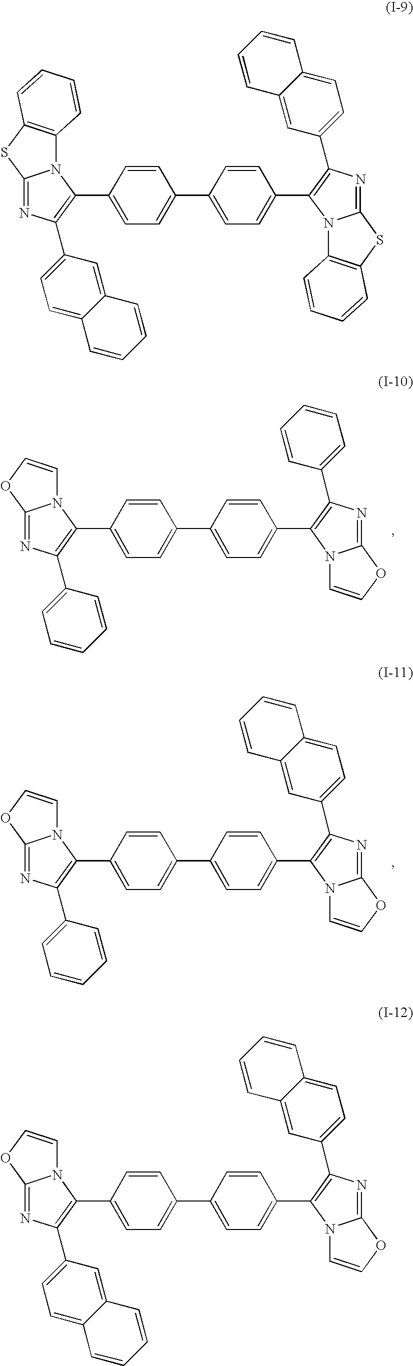 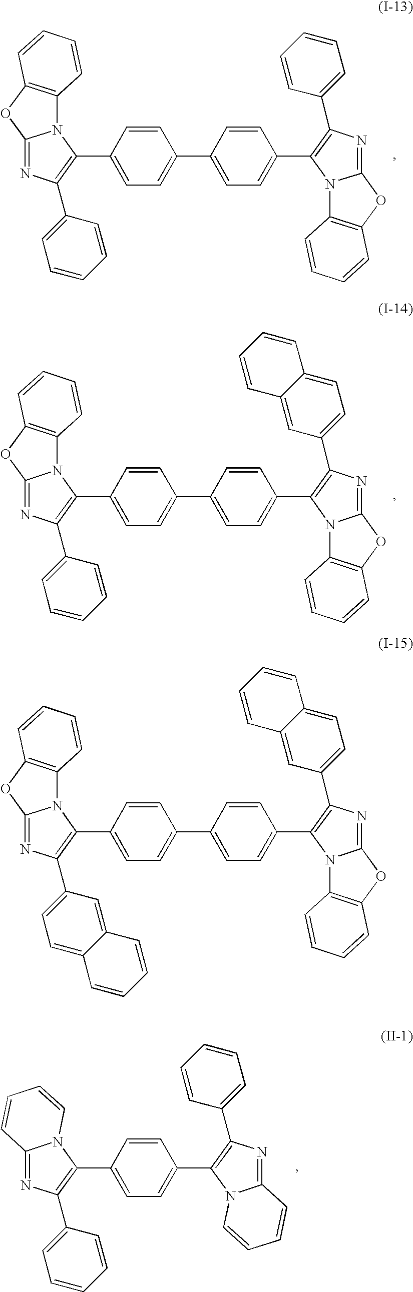 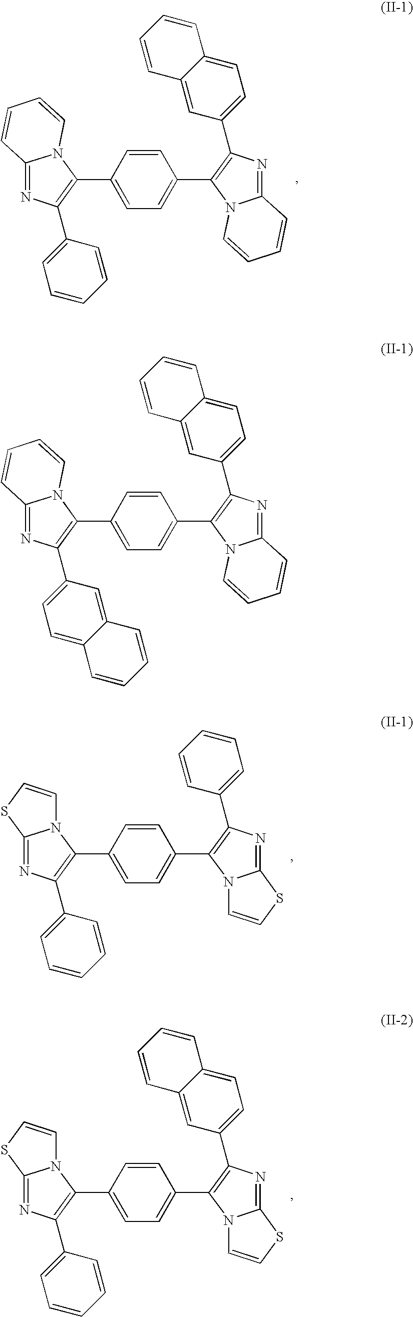 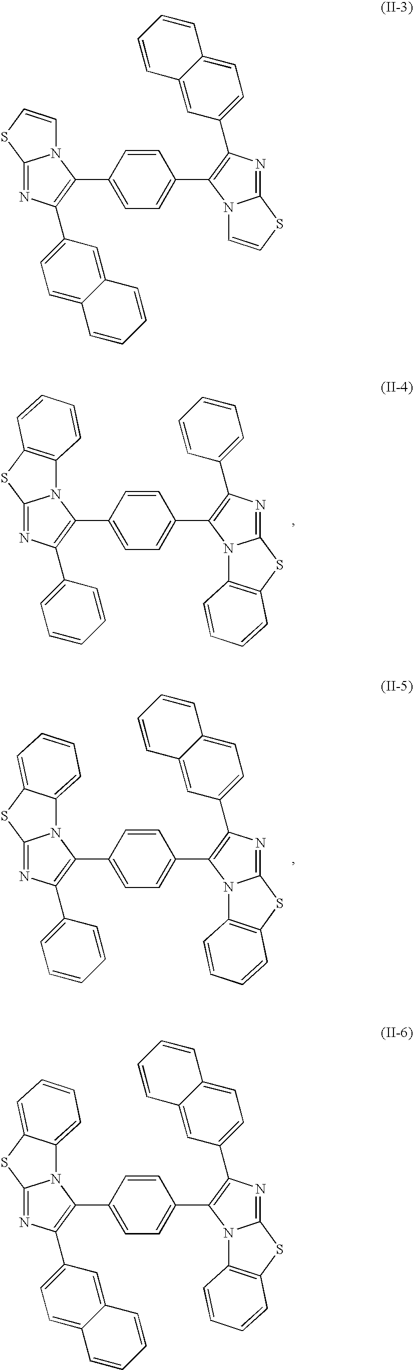 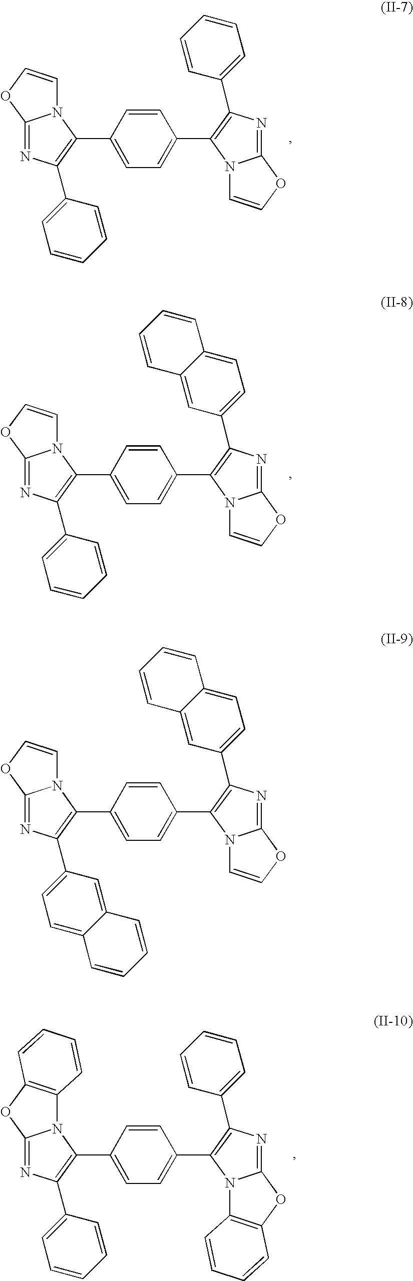 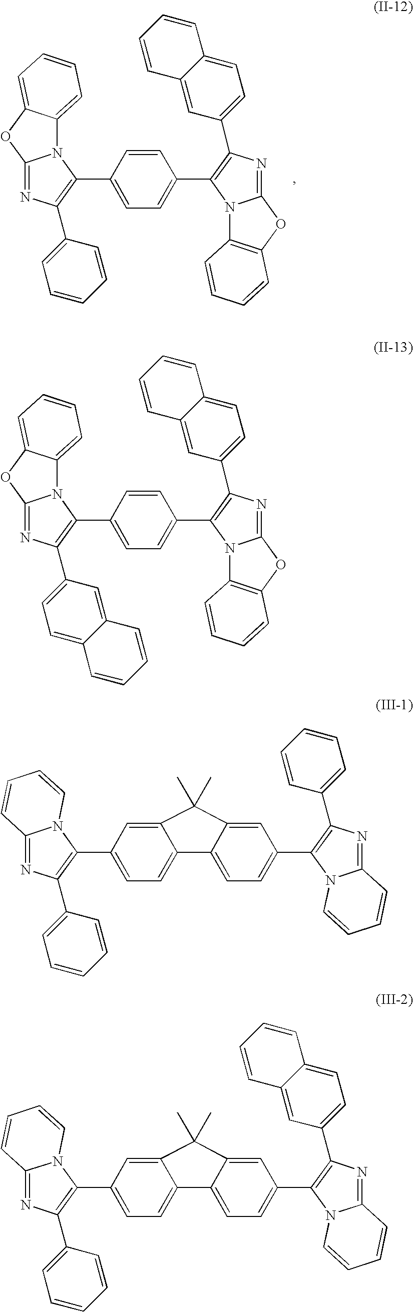 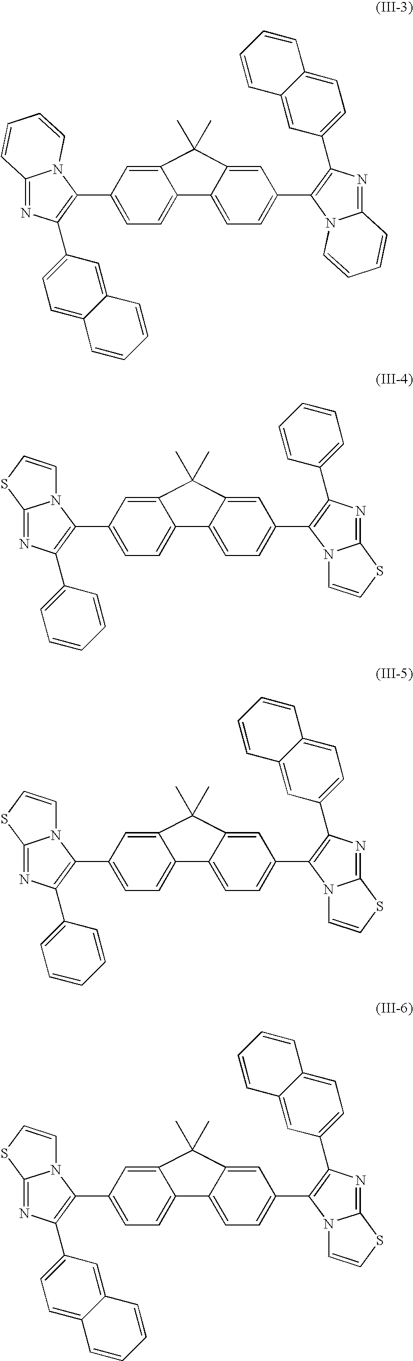 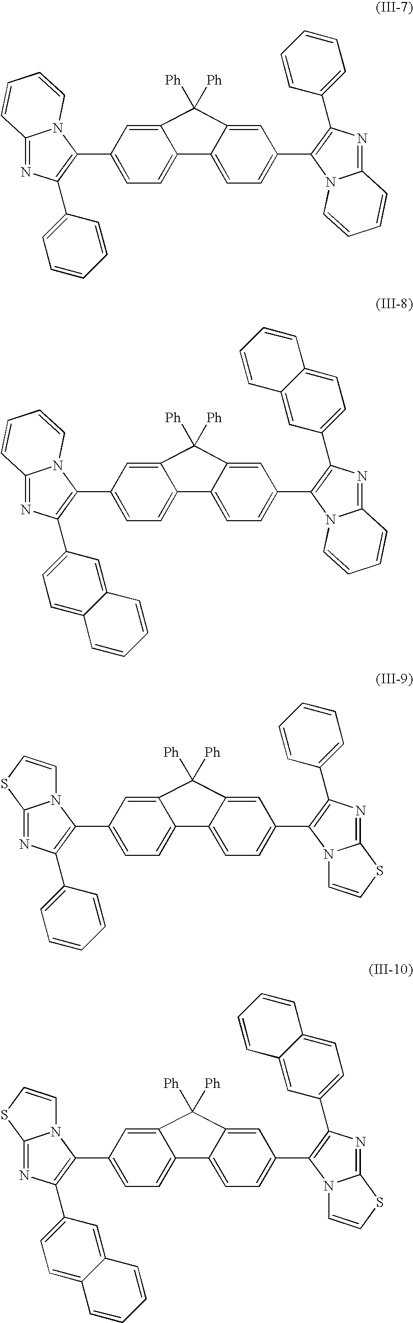 [chemical structure]
